(19) United States
(12) Patent Application Publication
(10) Pub. No.: US 2021/0383926 A1
(43) Pub. Date: Dec. 9, 2021

(54) MENTAL HEALTH ASSESSMENT SYSTEM AND METHOD

(71) Applicant: COGNIANT PTY LTD, Nedlands, WA (AU)

(72) Inventors: Mairin REID, Kallaroo, Western Australia (AU); Neeraj KOTHARI, Singapore (SG)

(73) Assignee: COGNIANT PTY LTD, OSBORNE PARK, WA (AU)

(21) Appl. No.: 16/770,330

(22) PCT Filed: Dec. 5, 2017
(86) PCT No.: PCT/AU2017/051337

§ 371 Date: Jun. 5, 2020

(30) Foreign Application Priority Data
Dec. 5, 2016 (AU) 2016905005

(51) Int. Cl. G16H 50/30; G16H 40/67; G16H 10/20; G16H 10/60; G16H 50/70; G16H 20/70

(52) U.S. Cl. CPC G16H 50/30; G16H 40/67; G16H 20/70; G16H 10/60; G16H 50/70; G16H 10/20

(57) ABSTRACT

A mental health assessment system comprises a mobile device comprising a plurality of sensors for receiving respective inputs over time; a feature monitoring module configured to interpret the sensor inputs as representing behaviours of a user of the mobile device; an analysis module for analysing the represented behaviours relative to external data to determine a risk measure; and a response module for responding to the behaviours according to the risk measure.

### FIELD OF THE INVENTION

The present invention relates to a method and system that assists in assessment and/or monitoring of mental health.

### BACKGROUND

Mental health has generally been a neglected area of public concern. Whilst there are mental health professionals that can assess a patient's mental health, this usually involves at least one, sometimes numerous, visits to the mental health professional and usually requires an “interview” to get to the bottom of the mental health problem. Further, between visits to the mental health professional there is no easy way of monitoring the mental wellbeing of the patient.

US 2013/0297536 describes a system for monitoring a user's mental health by tracking use of electronic devices, learning a unique behavioural pattern as a “base line” and using algorithmic processing to detect irregularities in behavioural patterns. The specific

US 2015/0313529 describes receiving from a mobile device sensor data and/or device state data, analysing the data to provide a behavioural pattern, comparing to a reference behavioural pattern and estimating a likelihood of an abnormal condition based on the comparison.

US 2015/0370994 describes modelling behaviour of a patient by receiving a log of use dataset associated with communication behaviour during a period of time; receiving a supplementary dataset characterising mobility-behaviour during the time-period (such as location information, movement information and device usage information); generating a predictive model based on a passive dataset derived from the log of use dataset and the supplementary dataset. The passive dataset and/or the predictive model output are transformed into an analysis.

The present invention seeks to provide a tool to assist in assessment and/or monitoring of mental health.

Any references to documents that are made in this specification are not intended to be an admission that the information contained in those documents form part of the common general knowledge known to a person skilled in the field of the invention, unless explicitly stated as such.

### SUMMARY OF THE INVENTION

According to the present invention there is provided a mental health assessment system, comprising:

a mobile device comprising a plurality of sensors for receiving respective inputs over time; a feature monitoring module configured to interpret the sensor inputs as representing behaviours of a user of the mobile device;
an analysis module for analysing the represented behaviours relative to external data to determine a risk measure;
a response module for responding to the behaviours according to the risk measure.

In an embodiment the behaviours comprise mobile device usage, movement of the mobile device. In an embodiment the analysis module also analyses user responses to prompting. In an embodiment the feature monitoring module comprises a user profile builder for building a user profile of user activity based on the represented behaviours.

In an embodiment the analysis module comprises a behaviour type selector for selecting a behaviour type based on the user profile.

In an embodiment the analysis module comprises a processor for determining the risk factor based on the selected behaviour type and the represented behaviours.

In an embodiment the feature monitoring module is implemented by the mobile device and the represented behaviours of the user are transmitted to a server which implements the analysis module. In an embodiment the external data is not used to determined features to be monitored by the feature monitoring module.

In an embodiment the risk measure is of a mood disorder.

Also according to the present invention there is provided a method of assessment of the mental health of a user of a mobile device comprising:

providing a plurality of sensors in mobile device for receiving respective inputs over time;
interpreting the sensor inputs as representing behaviours of a user of the mobile device;
analysing the represented behaviours relative to external data to determine a risk measure;
responding to the behaviours according to the risk measure.

Also according to the present invention there is provided a method of assessment of the mental health of a user of a mobile device comprising:

monitoring user activity by use of sensors of the mobile device;
building a user profile of user activity based on monitoring;
identifying a type of behavioural profile from the user's profile;
analysing current or recent activity to identify the user's risk based on the type of behavioural profile identified; and
responding to the user based on the user's identified risk

In an embodiment the monitoring comprises receiving data from the sensors and extracting data from logs of applications of the mobile device. In an embodiment the monitoring comprises one or more of: sleep pattern; phone use and/or duration; message sending pattern; time of use; medication taking compliance; location monitoring; movement monitoring; goal compliance.

In an embodiment the sensors comprise one or more of GPS; gyroscope; accelerometer; ambient light sensor; home button pressing; lock/unlock frequency; or input through a smartphone touch screen.

In an embodiment the building of the user's profile comprises determining patterns of activity of the user, the behaviours comprising mobile device usage, movement of the mobile device. In an embodiment the patterns of activity comprise one or more of travel to locations; erratic movement; sleeplessness; erratic sleep; insomnia; high amounts of communication compared to normal; failure to respond to messages.

In an embodiment the identifying the type of behavioural profile comprises correlation to a demographic profile. In an embodiment the identifying the type of behavioural profile comprises correlation to social profile. In an embodiment the identifying the type of behavioural profile comprises correlation to psychological disorders.

In an embodiment the analysis comprises identifying triggering events or activities where the user is at risk of mental unwellness. In an embodiment the mental unwellness is mental distress. In an embodiment the analysis comprises identifying symptoms of being at risk of being in a state of mental unwellness.

In an embodiment the response comprises sending a checking wellness message. In an embodiment the response comprises sending a reassurance message. In an embodiment the response comprises sending an alert. In an embodiment the response comprises providing a micro intervention.

Also according to the present invention there is provided a system for monitoring the mental health of a user, comprising

a monitoring means for monitoring user activity by use of sensors of the mobile device;
a user profile builder for building a user profile of user activity based on monitoring;
a behavioural type selector for identifying a type of behavioural profile from the user's profile;
a processor for analysing current or recent activity to identify the user's risk based on the type of behavioural profile identified and user response to prompting relative to external data; and
a response module for providing a response according to the analysis.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

### SUMMARY OF DRAWINGS

In order to provide a better understanding of the present invention, example embodiments will now be described with reference to the accompanying figures in which:

FIG. 1 is a system diagram of an embodiment of the present invention; and

FIG. 2 is a flow chart of a method of an embodiment of the present invention.

### DETAILED DESCRIPTION OF EMBODIMENTS OF THE INVENTION

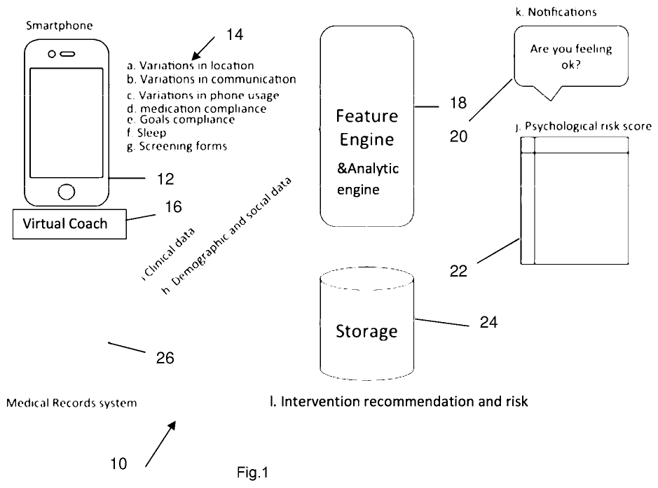

Referring to FIG. 1, there is a system 10 for assessing or monitoring mental health. The system 10 comprises a mobile device, such as a tablet computer, smartphone 12, smartwatch or other wearable device or devices. The mobile device 12 comprises a plurality of sensors for receiving respective inputs 14 over time. These inputs comprise: location sensors (such as Global Positioning Satellite (GPS) sensors), ambient light sensors, monitoring of input from the microphone, monitoring of camera images, inputs to the mobile device, such as home button activation and key logging, motion sensors (such as gyroscope and/or accelerometer). Further various logs from applications of the phone can be used as a “summary” of various sensor inputs, such as timing, of and duration of phone calls and messages.

These inputs from the sensors, can be collected over time by a computer program installed on the smart phone 12, which are commonly called an application, or simply an ‘app’. Generally this app runs in the background and periodically collects the inputs or accesses data stored by the smart phone 12 which represents or summarises the or some of the inputs.

The system 10 further comprises a feature monitoring module 18, which is configured to interpret the sensor inputs as representing behaviours of a user of the mobile device. These behaviours are not intended to be a diagnosis of a mental health issue. They are intended to be or be the equivalent of observations of symptoms. For example, location sensors track the extent of movement; ambient light sensors determine activity relative to time of day and brightness of the environment during activity; microphone input tracks volume and speed of speech; camera tracks eyeball movement; key logging tracks amount of typing; camera “selfie” categorization tracks the number of self-photographs in a time period and at a location. The feature monitoring module 18 may operate on the smart phone 12, or it may operate on a dedicated computing device in communication with the smart phone. In the latter case the inputs 14 are sent to the feature monitoring module 18, typically periodically. Further example of features based on location and time of day are: daily distance travelled, Max. distance travelled from home, time spent at home, weekday distance, weekend distance, and daily office commute distance.

When implemented on a dedicated computing device, the feature monitoring module 18 may be implemented so as to interpret the sensor inputs as representing behaviours of respective users of a plurality of mobile devices. Further the dedicated computing device may be configured to implement a plurality of feature monitoring modules in parallel, each for interpreting one or more of the sensor inputs as representing behaviours of respective users of a plurality of mobile devices. This will allow for monitoring of many users at once.

The module 18 interfaces with a medical records system 26 for receiving data from the medical records system 26. The medical records system 26 may be a medical records system used by a mental health professional or organisation, which stores information including clinical data with regard to the user of the mobile device and or demographic and social data, which may be stored in storage 24. The medical records may comprise results from a survey of the user. Alternatively the module may take into account user responses from prompting, such as compliance questions or survey questions. The survey may comprise PHQ-9 questions and/or Perceived Stress Scale questions. In an embodiment the module 18 interfaces with a weather information source for received data from the weather information source about weather conditions in the locality of the user.

As a separate module, or as part of the feature monitoring module 18, the system further comprises an analysis module for analysing the represented behaviours relative to external data, such as data from the medical records system 26, to determine a risk measure, represented by 22. The risk measure 22 is stored in storage 24. Storage 24 may be for example a hard disk drive or flash memory, which may be in the mobile device, in the dedicated computing device, networked to the computing device, or ‘cloud’ storage. In an embodiment the risk measure can be determined by deep phenotyping using neural networks on self reported survey data and input data 14 to nudge help seeking behavior in at risk population.

In an embodiment the feature monitoring module comprises a user profile builder for building a user profile of user activity based on the represented behaviours. The profile is preferably focused on building a help seeking behavior profile.

In an embodiment the analysis module comprises a behaviour type selector for selecting a behaviour type based on the user profile. The inputs are passive, which can be correlated with a patient subgroup with an ongoing condition to model and stratify patients' risk for behavioural conditions. Patient groups are classified based on this risk.

In an embodiment the analysis module comprises a processor for determining the risk factor based on the selected behaviour and the represented behaviours. The risk factor is preferably a representation of the risk that a user that have been diagnosed with a mood disorder by a medical professional is at risk of deviation from a treatment plan or is at risk of relapse. In an embodiment the risk factor is a mood state prediction and accounts for medical information about the predisposition of the user to enter (or has entered) a mental state based on the predicted mood.

As a separate module, or as part of the feature monitoring module 18, the system further comprises a response module for responding to the behaviours according to the risk measure. An example response is indicated by message 20. Further the response module may provide information to the health professional with regard to the behaviour and/or risk measure via the medical records system 26, so that the health professional has information about the mobile device's user in order to better diagnose and/or monitor treatment and/or to fine tune or adjust treatment, or as an indication of relapse or being at risk of relapse.

The response module can alert a care provider if risk score goes above the threshold or suggesting micro mobile interventions if the risk score is within certain threshold.

The dedicated computer implementing module 18 may be in the form of a server which comprises a memory, comprising volatile memory such as random access memory (RAM) and non-volatile memory, such as read only memory (ROM). The server comprises a computer program storage medium reader for reading the computer program instructions from computer program storage media. The storage media may be optical media such as DVD-ROM disks, magnetic media such as floppy disks and tape cassettes, hard disk drive, or flash media such as USB memory sticks.

The modules 18 be configured as electronic circuits or may be implemented by the processor of the server being configured by instructions of a computer program.

An example implementation is described below.
a. In an embodiment the feature module 18 detects variations in location data captured through GPS activities of Smartphone and motion sensors which detect routine movements to home, office and outlier movements.
b. In an embodiment the feature module 18 detects variation in communication based on call log frequency and message log frequency based on number of calls per day, time of call, and duration of call per day.
c. In an embodiment smartphone usage detection is determined by monitoring ambient light sensors in Smartphone and logging of lock/unlock frequency per use.
d. In an embodiment medication compliance is monitored by a virtual coach application 16 in the Smartphone app by sending user periodic reminders, either set by user or by care provider to take medication. Reminders are sent in the form of text message via the app which records response to the reminders and measures completeness of compliance via feature engine.
e. In an embodiment goal compliance is monitored by the virtual coach 16 in the smartphone app by sending user periodic reminders, either set by user or by care provider during the treatment. Reminders are sent in the form of text message via the app which records response to the reminders and measures completeness of compliance via feature engine.
f. In an embodiment the feature module 18 automatically detects the total duration of sleep via smartphone movements. Smartphone motion sensors are used to detect correlation between regular Smartphone usage and detect period of inactivity to determine the total daily sleep time.
g. In an embodiment screening forms are to be filled by smartphone user which can range from a simple one question Screener (Smoking habit) to multiple questions screening (e.g: PHQ9). These forms are clinical in nature and determine the psychological wellbeing of a person. In addition to detecting overall score of screening forms, the feature engine detects any new information captured in these survey forms by performing natural language processing for feature engineering.
h. The medical records system 26 used by health providers is interfaced with the use of application programming interface (API) to the feature engine and provides information on user socio-economic and demographic data.
i. The medical records system 26 also gives information about patient clinical data which includes, medications, problem history, other non psychological medical conditions like diabetes, asthma etc. and treatment.
j. In an embodiment the analytics engine picks up the features generated in feature engine and creates stratified psychological risk score 22 at patient level and population level, using k-means classification for patient clustering and predicting future behavior using single vector machine (SVM) classification model.
k. In an embodiment the analytics engine also determines the best time to send the user smartphone push notifications based on rules created through movement data and usage data. These rules are defined to encourage more physical activity based on negative entropy of movement data collected.
l. In an embodiment the risk score 22 is fed back to same or any other medical records system using API and this generates patient list for health provider to stratify the patient population from high risk to low risk and to intervene. The API also creates possible intervention options based on evidence of treatment and efficacy in the system.

Also according to the present invention there is provided method 100 of assessment of the mental health of a user of a mobile device comprising:

providing a plurality of sensors in mobile device for receiving respective inputs over time;
interpreting 104 the sensor inputs as representing behaviours of a user of the mobile device; analysing 108 the represented behaviours relative to external data to determine a risk measure;
responding 116-122 to the behaviours according to the risk measure.

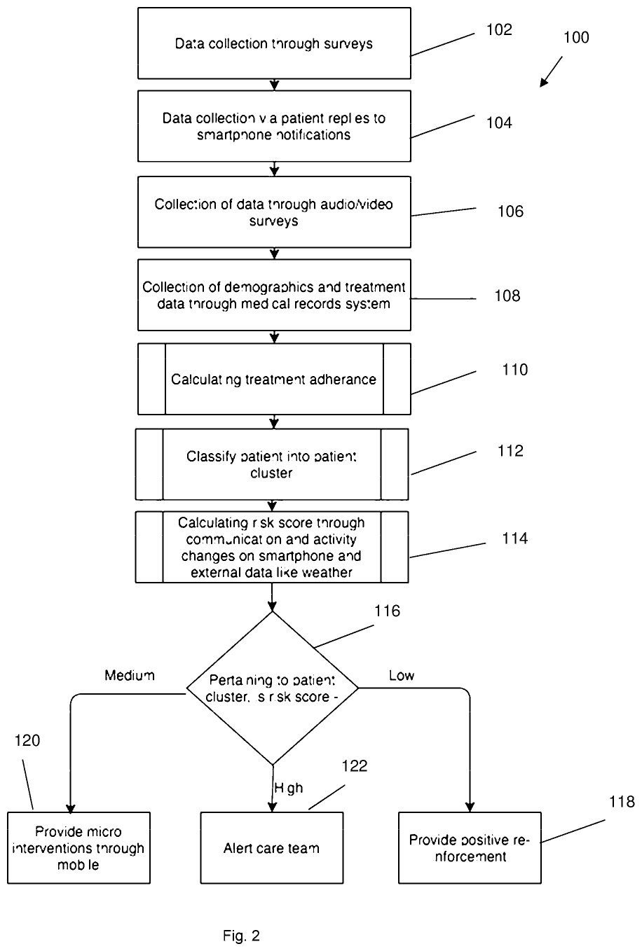

A more detailed example of the method 100 is described in relation to FIG. 2, which commences with collecting data through surveys 102. The surveys ask participants questions relevant to their mental health. Replies to smart phone notifications are collected at 104. Data is collected 106 through audio/video surveys. Demographics and treatments data are collected 108 through medical records systems. From the data collected at 102, 104, 106 and 108 treatment adherence is calculated at 110. The/each patient is classified 112 into a patient cluster. A risk score is calculated 114 through communication and activity changes on smartphone and external data, such as weather. The risk score for the patient cluster determined 116 the next course of action. If the risk is low then positive reinforcement is provided 118. If the risk is medium then micro interventions are provided 120 through the smart phone. If the risk is high then an alert is sent 122 to a care team.

FIG. 2 describes the method for collection of above mentioned data and generating risk score for individual users. These scores are compared against individual cluster thresholds to determine the suitable interventions for the user.
1. In an embodiment the method comprises notifying a care provider based on the risk score of a user and providing micro behavioral interventions centered on principles of mindfulness, resilience, peer support, gratefulness and cognitive behavioral therapy.
2. In an embodiment the method comprises creating alerts and notifications based on cluster of population and the rules defined under feature engine for positive behavior encouraging them to go on a walk or to do a meditation or to record their thoughts.
3. In an embodiment the method comprises creating treatment efficacy using medication and goals compliance and using that to inform care providers.
4. In an embodiment the method comprises using text, voice samples, audio, weather data to quantify and monitor daily mood and using that in feature engine as input to the risk score.
5. In an embodiment the method comprises using patient demographics, socio-economic indicators, clinical history collected through survey forms to stratify patient population into different clusters and comparing these clusters with variations in smartphone usage, phone call frequency and duration, text frequency, location changes, mobile phone and app usage.

Modifications may be made to the present invention within the context of that described and shown in the drawings. Such modifications are intended to form part of the invention described in this specification.

1. A mental health assessment system, comprising:
a mobile device comprising a plurality of sensors for receiving respective inputs over time;
a feature monitoring module configured to interpret the sensor inputs as representing behaviours of a user of the mobile device;
an analysis module for analysing the represented behaviours relative to external data to determine a risk measure;
a response module for responding to the behaviours according to the risk measure.

2. A system as claimed in claim 1, wherein the behaviours comprise mobile device usage, movement of the mobile device.

3. A system as claimed in claim 1 or 2, wherein the analysis module also analyses user responses to prompting.

4. A system as claimed in any one of claims 1 to 3, wherein the feature monitoring module comprises a user profile builder for building a user profile of user activity based on the represented behaviours.

5. A system as claimed in any one of claims 1 to 4, wherein the analysis module comprises a behaviour type selector for selecting a behaviour type based on the user profile.

6. A system as claimed in any one of claims 1 to 5, wherein the analysis module comprises a processor for determining the risk factor based on the selected behaviour type and the represented behaviours.

7. A system as claimed in any one of claims 1 to 6, wherein the feature monitoring module is implemented by the mobile device and the represented behaviours of the user are transmitted to a server which implements the analysis module.

8. A system as claimed in any one of claims 1 to 7, wherein the external data is not used to determined features to be monitored by the feature monitoring module.

9. A method of assessment of the mental health of a user of a mobile device comprising:
providing a plurality of sensors in mobile device for receiving respective inputs over time;
interpreting the sensor inputs as representing behaviours of a user of the mobile device;
analysing the represented behaviours relative to external data to determine a risk measure;
responding to the behaviours according to the risk measure.

10. A method of assessment of the mental health of a user of a mobile device comprising:
monitoring user activity by use of sensors of the mobile device;
building a user profile of user activity based on monitoring;
identifying a type of behavioural profile from the user's profile;
analysing current or recent activity to identify the user's risk based on the type of behavioural profile identified; and
responding to the user based on the user's identified risk

11. A method as claimed in claim 10, wherein the analysis comprises analysing user responses to prompting.

12. A method as claimed in claim 10 or 11, wherein the monitoring comprises receiving data from the sensors and extracting data from logs of applications of the mobile device.

13. A method as claimed in any one of claims 10 to 12, wherein the monitoring comprises one or more of: sleep pattern; phone use and/or duration; message sending pattern; time of use; medication taking compliance; location monitoring; movement monitoring; goal compliance.

14. A method as claimed in any one of claims 10 to 13, wherein the building of the user's profile comprises determining patterns of activity of the user, the behaviours comprising mobile device usage, movement of the mobile device.

15. A method as claimed in claim 14, wherein the patterns of activity comprise one or more of travel to locations; erratic movement; sleeplessness; erratic sleep; insomnia; high amounts of communication compared to normal; failure to respond to messages.

16. A method as claimed in any one of claims 10 to 15, wherein the identifying the type of behavioural profile comprises correlation to a demographic profile.

17. A method as claimed in any one of claims 10 to 16, wherein the identifying the type of behavioural profile comprises correlation to social profile.

18. A method as claimed in any one of claims 10 to 17, wherein the analysis comprises identifying triggering events or activities where the user is at risk of mental unwellness.

19. A method as claimed in any one of claims 10 to 18, wherein the analysis comprises identifying symptoms of being at risk of being in a state of mental unwellness.

20. A method as claimed in any one of claims 10 to 19, wherein the response comprises sending a checking wellness message.

21. A method as claimed in any one of claims 10 to 20, wherein the response comprises sending a reassurance message.

22. A system for monitoring the mental health of a user, comprising
a monitoring means for monitoring user activity by use of sensors of the mobile device;
a user profile builder for building a user profile of user activity based on monitoring;
a behavioural type selector for identifying a type of behavioural profile from the user's profile;
a processor for analysing current or recent activity to identify the user's risk based on the type of behavioural profile identified; and
a response module for providing a response according to the analysis.
